(19) United States
(12) Patent Application Publication
(10) Pub. No.: US 2013/0072516 A1
(43) Pub. Date: Mar. 21, 2013

(54) METHOD OF ALCOHOL CESSATION AND TREATMENT

(71) Applicants: James William Carpenter, Lake Orion, MI (US); Ricardo D. Borrego, Grosse Pointe Farms, MI (US)

(72) Inventors: James William Carpenter, Lake Orion, MI (US); Ricardo D. Borrego, Grosse Pointe Farms, MI (US)

(73) Assignee: EAGLE ADVANCEMENT INSTITUTE LLC, West Bloomfield, MI (US)

(21) Appl. No.: 13/607,652

(22) Filed: Sep. 7, 2012

Related U.S. Application Data
(60) Provisional application No. 61/531,753, filed on Sep. 7, 2011.

(51) Int. Cl. A61K 31/495; A61K 31/485; A61K 31/16; A61K 31/10

(52) U.S. Cl. CPC A61K 31/485; A61K 31/16
USPC 514/282

(57) ABSTRACT

A method of alcohol cessation and treatment for a patient generally comprises the steps of administering a multi-vitamin to the patient, administering a medication group to the patient, and monitoring the patient. In various embodiments, the medication group may be administered to the patient via a first and second injection solution. The first injection solution generally comprises a therapeutically effective amount of an anti-addictive agent, such as naltrexone palmitate. The second injection solution generally comprises a therapeutically effective amount of an inhibitor of alcohol dehydrogenase, such as disulfiram. The method, which may also be referred to as procedure, may include various pre- and/or post-procedural steps as described herein.

### CROSS REFERENCE TO RELATED APPLICATIONS

This application claims the benefit of U.S. Provisional Patent Application Ser. No. 61/531,753 filed on Sep. 7, 2011, which is incorporated herein by reference in its entirety.

### FIELD OF THE INVENTION

The present invention generally relates to a method of alcohol cessation and treatment.

### DESCRIPTION OF THE RELATED ART

Alcohol addiction (or alcoholism) is a state of periodic or chronic intoxication produced by the repeated consumption of alcohol. Alcoholism is a disabling brain disorder, and is medically defined as a treatable disorder. Long term alcohol abuse by a user (i.e., an alcoholic) produces physiological changes in the brain such as tolerance and dependence. Brain chemistry changes maintain the alcoholic's compulsive inability to discontinue drinking alcohol and can result in severe alcohol withdrawal syndrome upon discontinuation of alcohol intake. Due to its cumulative toxic effects, alcohol generally damages all bodily organs of the alcoholic, including the brain.

Alcoholism is generally the cyclical presence of tolerance, withdrawal, and continued excessive use in the presence of psychological, physiological, and interpersonal issues. Characteristics of alcoholism can include one or more of the following: compulsion to continue drinking alcohol; inability to stop drinking independently; interpersonal, school or work-related problems directly attributable to alcohol use; physical ailments, such as liver cirrhosis and hepatitis, pancreatitis, peripheral neuropathy, and/or cardiomyopathy; trauma, such as bone fractures, fatigue, depression, sexual dysfunction, fluctuating blood pressure, and/or sleep disorders; and severe withdrawals, such as seizures and delirium tremens (DTs), which can include severe agitation, diarrhea, confusion, hallucinations, fever, and/or uncontrollable tremors.

There are five main neurotransmitters affected by alcoholism. Two of these chemicals, dopamine and serotonin, have a profound affect upon the alcoholic. These two neurotransmitters affect the alcoholic by changing how they respond under stress, what moods are experienced, and can also communicate feelings of pleasure and/or pain. Chronic alcohol use affects the production of these neurotransmitters in the following ways. When dopamine is depleted by alcohol use, it can leave a user with a high tolerance to pain and reduced level of pleasure one feels when doing an activity. When serotonin is depleted by alcohol use, it can lead to sensory deprivation, body temperature anomalies, and/or depression.

Similar to alcohol addiction, even the occasional use (or abuse) of alcohol impairs a user's ability to achieve goals and fulfill responsibilities. Relative to addiction, mere alcohol abuse is generally not characterized by incessant cravings for alcohol, or total loss of control. In this way, an abuser may sometimes (over)indulge in alcohol use chronically; however, when not doing so, the abuser will typically not suffer serious physical withdrawal symptoms. In general, an abuser is one that drinks despite social, impersonal, legal and other recurrent problems that arise out of his/her social drinking.

An abuser generally demonstrates a pattern of excessive drinking and usually sets out to drink in order to feel the “high” associated with it. Other common alcohol abuse symptoms can include one or more of the following: often turning up late for work and/or school due to hangovers; frequent injuries that occur during times of excessive drinking; trouble with the authorities for drinking and driving (e.g. driving under the influence or “DUI”; driving while visibly intoxicated/impaired or “DWVI”; or reckless driving); aggressive behavior; drunkenness, drowsiness, and/or slurred speech; short attention span; uncoordinated movements; memory problems; continuing to drink in the face of interpersonal relationship problems; binge drinking, which is continuous heavy drinking over the course of a few hours to several days; legal problems; drinking in dangerous situations; and failure to assume responsibilities for work, school, etc.

There are various ways for an alcoholic or abuser to stop the use of alcohol. Completely stopping the use of alcohol is an ideal goal of treatment, which is called abstinence. A strong social network and family support are important in achieving this. However, completely stopping and avoiding alcohol is extremely difficult for alcoholics and chronic abusers.

Alcohol recovery or support programs can help alcoholics and abusers stop drinking completely. These programs usually offer: counseling and therapy to discuss alcoholism and its effects and how to control thoughts and behaviors; mental health support; and medical care. Alcoholics Anonymous (“AA”) is just one example of such a support group. Unfortunately, such programs are voluntary. As such, an alcoholic or abuser can choose to ignore the assistance provided by such programs, even in the face of legal ramifications.

Once abstinence is generally started, medications are sometimes prescribed to prevent drinking from occurring again. Three main medications for such a purpose include acamprosate (often branded as Campral®), disulfiram (often branded as Antabuse®), and naltrexone (often branded as Vivitrol®).

Acamprosate is typically used along with counseling and social support to help users who have stopped drinking large amounts of alcohol (e.g. alcoholics) to avoid drinking alcohol again. As described above, drinking alcohol for a long time changes the way the brain works. Acamprosate works by helping the brains of users who have drunk large amounts of alcohol to work normally again. Conventional acamprosate comes as a delayed-release tablet, taken by mouth. It is usually taken three times a day. Unfortunately, acamprosate does not prevent withdrawal symptoms that users may experience when they stop drinking alcohol. In addition, acamprosate has not been shown to work in users who have not completely stopped drinking alcohol or in users who drink large amounts of alcohol.

Alternatively, disulfiram is typically used to treat chronic alcoholism. It causes unpleasant effects when even small amounts of alcohol are consumed. These effects can include flushing of the face, headache, nausea, vomiting, chest pain, weakness, blurred vision, mental confusion, sweating, choking, breathing difficulty, and/or anxiety. These effects begin about 10 minutes after alcohol enters the user's body and can last for one or more hours. Conventional disulfiram comes in the form of tablets, taken by mouth. It is usually taken once a day. Unfortunately, while disulfiram can discourage drinking in its oral (per os or “PO”) form, it is not a cure for alcohol cessation.

Alternatively, naltrexone injection is typically used along with counseling and social support to help users who have stopped drinking large amounts of alcohol to avoid drinking again. In general, naltrexone injection should not be used to treat users who are still drinking alcohol. Naltrexone is in a class of medications called opiate antagonists. It works by blocking activity in the limbic system, a part of the brain that is involved in alcohol dependence. Naltrexone injection comes as a solution (liquid) to be given by injection into muscle, e.g. muscle of the buttocks, by a healthcare provider. The injection is usually given once every four weeks. Unfortunately, naltrexone injection will not prevent withdrawal symptoms that may occur when users stop drinking alcohol after drinking large amounts for a long time.

While there are programs and drugs that can help an alcoholic or abuser, many, if not all of them suffer from one of more deficiencies as described above. As such, there remains an opportunity to provide improved methods for alcohol cessation and treatment.

### SUMMARY OF THE INVENTION AND ADVANTAGES

The subject invention provides a method for alcohol cessation and treatment. In one embodiment, the method comprises the steps of administering a multi-vitamin to the patient, administering a medication group to the patient, and monitoring the patient. In certain embodiments, the medication group is administered to the patient via a first and second injection solution.

The method of the present invention provides excellent cessation results while also generally reducing pain and discomfort associated with conventional methods. The method of the present invention is especially suitable for alcohol abusers, where withdrawal symptoms are less of an issue relative to alcoholics. The method of the present invention has excellent results, such as preventing future alcohol use or abuse by a user/patient. The method generally enables patients to effectively prevent continued alcohol use with successful cessation, and is a long term deterrent from further alcohol abuse by the patient.

### DETAILED DESCRIPTION OF THE INVENTION

The subject invention provides a method (hereinafter referred to as the “procedure”) for alcohol cessation and treatment of a patient. The patient can be an alcohol abuser or an alcoholic. Typically, the patient is an alcohol abuser, i.e., the patient is not an alcoholic. Alcoholics can have complicated withdrawal issues, and typically should have some form of mitigation beforehand. The procedure can be utilized at various intervals, such as once a month, while the patient is undergoing cessation treatment. In general, the invention procedure will mitigate cravings and deter future alcohol use/abuse by the patient.

The procedure can be administered in various locations and by various individuals. For example, the procedure can be administered in a clinically equipped medical/surgical environment by licensed and trained physician and/or medical staff.

The procedure of the present invention generally includes the steps of: 1) administering a multi-vitamin to the patient; 2) administering a medication group to the patient; 3) monitoring the patient; and, optionally, 4) administering a sedative to the patient.

One or more pre-procedural steps may also be utilized before the procedure itself (i.e., before steps 1) through 3)/4)) of the invention procedure. The procedure is described further below. If employed, the pre-procedural step(s) is/are typically conducted a day or more prior to the procedure. The pre-procedural step(s)/preparation(s) can include one or more of the following steps described immediately below. It is to be appreciated that in certain embodiments, the invention procedure further includes one or more of the pre-procedure steps.

### Pre-Procedure:

The pre-procedural preparation(s) can include determining immediate blood alcohol level of the patient. Such levels generally should be negligible (i.e., at or approaching zero) before proceeding with the procedure. If blood alcohol level is elevated (i.e., greater than zero, greater than 0.01, greater than 0.02, etc.), recommendations can be made to address immediate treatment and the patient can be advised to return when successful abstinence has been attained. The patient may require professional intervention. The patient generally undergoes a written and visual assessment on site, e.g. at a treatment facility, to determine an accurate degree of addiction/abuse. Each of the aforementioned steps can be used alone, or in various combinations with one another.

The pre-procedural preparation(s) can also include, in addition or alternate to one of more of the step(s) above or below, a history and physical of the patient, including: current medical history of the patient; identification and demographics of the patient, such as name, age, height, and weight; past medical history of the patient, including major illnesses, surgeries/operations, current and/or chronic illnesses; family or childhood illnesses of the patient, and previous personal and/or family history of problems associated with delivery of anesthesia; social history of the patient, such as tobacco and alcohol use; drug history of the patient, such as regular and acute medication use, including over the counter (“OTC”), prescribed, herbal medicines and therapies, and history of opiate use both prescribed and recreational; and drug allergies of the patient. Each of the aforementioned steps can be used alone, or in various combinations with one another.

The pre-procedural preparation(s) can also include, in addition or alternate to one or more of the step(s) above or below, gathering insurance information for the patient in the event that the patient requires hospital attention. Blood can be drawn from the patient for lab evaluation including blood alcohol level. Based on the patient's previous medical and physical evaluations, cardiac evaluation may be warranted and an electrocardiography (“ECG”) and/or a stress test may be recommended. Each of the aforementioned steps can be used alone, or in various combinations with one another.

The pre-procedural preparation(s) can also include, in addition or alternate to one or more of the step(s) above or below, psychological and medical evaluations. Psychological evaluation can be done by a qualified addiction treatment therapist and all medical information can be reviewed. Post procedure instructions for family/friend caregiver of the patient can be reviewed. Medications for the patient can be drawn up and labeled for the invention procedure. A physical examination of the patient can be performed. Each of the aforementioned steps can be used alone, or in various combinations with one another.

The pre-procedural preparation(s) can also include, in addition or alternate to one or more of the step(s) above or below, a patient assessment, such as with a physician. The physician can perform an individual, personal interview with the patient designed to verify previously obtained information useful in formulating adequate anesthesia and a recovery plan. Review can include, but is not limited to, all of patient information described above. Neurological clinical examination of the patient can be conducted, including consciousness, awareness, brain, vision, and others indicated by physician. Each of the aforementioned steps can be used alone, or in various combinations with one another.

The pre-procedural preparation(s) can also include, in addition or alternate to one or more of the step(s) above or below, a general description of the procedure to the patient. All available data can be reviewed with the patient, with reference to patient history, and description of the anticipated procedure by the physician with the patient to confirm the patient's and/or legal representative's understanding of the planned procedure. All lab test results and any additional required tests can be reviewed by an interviewing physician. Each of the aforementioned steps can be used alone, or in various combinations with one another.

Prior to the procedure, various tests can be conducted, in addition or alternate to one or more of the step(s) above or below. Typically, the tests are performed the day of the procedure. Such tests can include, but are not limited to, an alcohol urine test; a breathalyzer test; a baseline blood saturation level of disulfiram (or a metabolite thereof) with an analyzer, such as a Zenalyser® (commercially available from ZenaMed Ltd. of Stoke-on-Trent, England); a urine β-hCG test, which should be done for all women of childbearing age; a drug test, such as a 7 panel test; and vital signs of the patient to establish a baseline of the patient. Each of the aforementioned tests or steps can be used alone, or in various combinations with one another.

In various embodiments, a Zenalyser® is used with the invention method. The Zenalyser® is a hand-held breathalyzer capable of detecting disulfiram breakdown products. As an example, if 200 milligrams (mgs) of disulfiram is consumed daily by a patient, the Zenalyser® is typically both 100% specific and 100% sensitive in detecting disulfiram breakdown products (with some margin of error in certain patients). Typically, the Zenalyser® works by measuring metabolite gases of disulfiram in the patient's breathe. As such, it has been found that using the Zenalyser® is very effective in confirming compliance or effectiveness of disulfiram treatments, e.g. the disulfiram injection(s) as described further below.

Other suitable analyzers for determining levels of disulfiram, or a metabolite thereof, for purposes of the present invention, are described in U.S. Pat. No. 5,802,909 to Faulder et al., U.S. Pat. No. 6,248,078 to Risby et al., U.S. Pat. No. 6,748,792 to Freund et al., and U.S. Patent Application Publication No. 2008/0314115 to Faulder et al., which are incorporated herein by reference in their entirety to the extent they do not conflict with the general scope of the present invention. The invention procedure will now be described.

### Procedure:

As first introduced above, a multi-vitamin is typically administered to the patient. The multi-vitamin can comprise various types of vitamins, and can be tailored to specific needs/traits of the patient, such as gender, age, overall health, etc. Typically, the multi-vitamin is administrated intravenously, such that the multi-vitamin is a multi-vitamin solution (which can be of various concentrations). The multi-vitamin can be in the form of a saline solution. The multi-vitamin can be administered in various amounts. The amount of the multi-vitamin can be tailored to specific needs/traits of the patient, such as gender, age, weight, overall health, etc. The multi-vitamin is useful for replenishing depleted vitamin levels in the patient.

Optionally, a sedative can be administered to the patient, if the patient so desires. In certain embodiments, the sedative is utilized, whereas in others, the sedative is not. The sedative can comprise various sedatives, and can be tailored to specific needs/traits of the patient, such as gender, age, weight, overall health, etc. The sedative can be in various forms and be of various types. Typically, the sedative is administered intravenously (and can be of various concentrations). The sedative can be in the form of a saline solution. The sedative can be administered in various amounts. The amount of the sedative can be tailored to specific needs/traits of the patient, such as gender, age, weight, overall health, etc. In certain embodiments utilizing the sedative, the sedative comprises midazolam. The midazolam can be administered, e.g. intravenously, in various amounts, such as about 1 mg/ml in a dose of from about 1 to about 2 mg. If utilized, the sedative is useful for calming the patient during the procedure.

After (or while/during) administering the multi-vitamin, and, optionally, the sedative, a medication group is administered to the patient. Typically, the medication group comprises naltrexone and disulfiram (hereinafter collectively referred to as the “N & D”), such that the N & D is administered to the patient. Naltrexone may be referred to in the art as an anti-addictive agent. Disulfiram may be referred to in the art as an inhibitor of alcohol dehydrogenase. The N & D can be administered simultaneously, or one before the other (in either order). Each of the N & D are typically in an injectable form, e.g. in an injection solution form. It is to be appreciated that this injectable form of the disulfiram is different than conventional forms of disulfiram, which are typically in pill or polymeric (implant) forms. As such, both of the N & D can be administered intramuscularly. The N & D injection solutions can be administered in various muscle locations, such as in the patient's buttocks (i.e., gluteus muscle) or the patient's thigh (i.e., quadriceps muscle). One or more muscle locations may be utilized. In certain embodiments, the N & D are injected separately as first and second injection solutions.

Each of the N & D injection solutions can be in various concentrations (or strengths). For example, the naltrexone injection solution can be a naltrexone palmitate injection solution having a concentration of about 200 mg/ml. Other concentrations can also be used, such as, but not limited to, naltrexone hydrochloride injection solutions in concentrations ranging from about 1 mg/ml to about 10 mg/ml. Typically, each of the N & D injection solutions is a “suspension” of the respective medication in normal saline or an oil, such as cottonseed oil, which may also be referred to in the art as a “slurry”. Typically, each of the N & D injection solutions is administered separately as an intramuscular injection, e.g. as first and second injection solutions.

Suitable disulfiram injection solutions, for purposes of the present invention, are available from a variety of suppliers. An example of a suitable disulfiram injection solution, for purposes of the present invention, is described in U.S. Pat. No. 4,678,809 to Phillips, which is incorporated herein by reference in its entirety to the extent it does not conflict with the general scope of the present invention. Suitable naltrexone injection solutions, for purposes of the present invention, are available from a variety of suppliers. A specific example of a suitable supplier for suitable naltrexone injection solutions (e.g. naltrexone palmitate 200 mg/ml injection solutions) is Wedgewood Pharmacy of Swedesboro, N.J. Another example of a suitable injection solution comprising both disulfiram and naltrexone, for purposes of the present invention, is described in U.S. Patent Application Publication No. 2010/0311704 to Gooberman, which is incorporated herein by reference in its entirety to the extent it does not conflict with the general scope of the present invention. The concentrations of each of the N & D injection solutions can be tailored to specific needs/traits of the patient, such as gender, age, weight, overall health, etc. Typically, the naltrexone and disulfiram are utilized in therapeutic amounts.

In certain embodiments, the disulfiram is prepared in a cottonseed oil preparation, such as 1 mg disulfiram per 2 ml of cottonseed oil. Both injections of N & D typically last greater than 30 days. As such, no oral pill is required. If necessary, monthly follow-up injections can be utilized for ongoing alcohol cessation and treatment. In certain embodiments, as introduced above, the Zenalyser® can be utilized to ensure efficacy and duration of the disulfiram injection.

The N & D injection solutions can be administered in various amounts. The amounts of each of the N & D injection solutions can be tailored to specific needs/traits of the patient, such as gender, age, weight, overall health, etc., as well as the individual concentrations of each of the N & D injection solutions.

In a specific embodiment, about 500 mg of naltrexone palmitate injection solution (e.g. about 500 mg per about 2 to about 2.5 ml of a cottonseed oil solution) is administered to the patient intramuscularly to block the effects of alcohol and control alcohol cravings. Further, about 1 to about 2 grams of disulfiram injection in about 2 to about 5 ml cottonseed oil solution is administered to the patient intramuscularly to prevent the subsequent use of alcohol after the procedure. The naltrexone can be administered before, after, or at the same time as the disulfiram is administered. Typically, the naltrexone is administered before the disulfiram. As described above, both the N & D are administered intramuscularly, e.g. as first and second injection solutions.

After injection into the patient, each of the N & D is released from the injection site(s) in a sustained-release manner. Without being bound by any particular theory, it is believed that a synergy exists being the N & D, especially while in an injectable form, which promotes expedited alcohol cessation and recovery of the patient. Since both the N & D are in the patient's body, and will be present for some amount of time after injection (e.g. over several weeks), the patient either has no desire to drink, or if he/she does, is not able to “cheat” by drinking alcohol without feeling ill. Said another way, in the present invention, the patient cannot simply just choose to not take his/her medications (as in the case with conventional methods relying on pill/oral forms of treatment), and thus relapse with alcohol abuse. Instead, the patient is continuously subjected to cessation treatment over the course of many weeks and months. In certain embodiments, the Zenalyser® can be utilized to ensure efficacy and duration of the ongoing treatment.

Unlike the invention procedure, conventional treatment methods require a pill to be taken to assure medications for alcohol cessation reach adequate blood levels to prevent and treat alcohol consumption. These treatments are patient driven and can be ceased at anytime (e.g. by the patient or by another) causing a void in the treatment of alcohol cessation. The procedure of the present invention solves this problem by administering the N & D which maintains therapeutic blood levels of independently proven medications to prevent alcohol consumption for at least 30 days (and beyond) with monthly follow-ups.

During the N & D administration, the multi-vitamin administration can be continuous or be stopped. Typically, during administration, the patient's vitals (or vital signs) are continuously monitored such as, but not limited to, the patient's blood pressure, ECG, SaO₂, and/or respirations.

After the N & D is administered, administration of the multi-vitamin is ceased (if still on-going), and the patient is typically monitored for an amount of time thereafter, such as about one hour. The patient can be monitored for more or less time. Typically, the patient's vitals are monitored and assessed for at least one hour after the procedure. In general, if all vital signs of the patient are stable while meeting certain discharge criteria, the patient can be released to return home. The discharge criteria can be tailored to specific needs/traits of the patient, such as gender, age, weight, overall health, etc.

In general, the invention procedure lasts about one hour; however, the procedure may last for more or less time. Afterwards, the patient is free to return home with recommendations for continuing care and returning to normal activities. These recommendations can be tailored to specific needs/traits of the patient, such as gender, age, weight, overall health, etc.

### Post-Procedure:

One or more post-procedural steps may also be utilized after the procedure itself (i.e., after steps 1) through 3)/4)) of the invention procedure. If employed, the post-procedural step(s) is/are typically conducted the same day of or soon after the procedure. The post-procedural step(s) can include one or more of the following steps described immediately below. It is to be appreciated that in certain embodiments, the invention procedure further includes one or more of the post-procedure steps. The post-procedure step(s) can be used alone or in combination with the pre-procedure step(s). Likewise, the pre-procedure step(s) can be used alone or in combination with the post-procedure step(s).

Recommendations for continuing care can be discussed and reviewed with the patient and a caregiver of the patient. Evaluation or post-procedure recommendations for necessary anxiolytics/benzodiazepine can be discussed with the patient and if needed, prescriptions can be written for the patient. The patient can be discharged alongside a caregiver with recommendations for continuing monthly intramuscular injections, multivitamin prescriptions, and/or recommendations for continuing care. Scheduled dates for subsequent monthly intramuscular injections are typically confirmed prior to discharge of the patient. It is believed that long term success rates are higher with continued treatment (e.g. monthly intramuscular injections of the N & D injection solutions) and psychological counseling of the patient. Each of the aforementioned steps can be used alone, or in various combinations with one another. In certain embodiments, the Zenalyser® can be utilized to ensure efficacy and duration of the ongoing treatment.

The following examples, illustrating the procedure, as well as of the pre- and post-procedures, of the present invention, are intended to illustrate and not to limit the invention.

### EXAMPLES

As described above, the invention method provides a low-risk, effective monthly intramuscular injection program for patients with alcohol abuse issues. The program typically enables patients to effectively prevent continued alcohol use with successful cessation, and is a long term deterrent from further alcohol abuse. Generally administered in a clinically equipped medical/surgical environment by licensed and trained physician and medical staff, the procedure includes one or more monthly medication injections which will mitigate cravings and deter future alcohol use/abuse.

Under the care of a licensed and trained physician, an intravenous line is started with a multi-vitamin preparation. A mild sedative is offered to the patient. Intramuscular injections of the naltrexone and disulfiram are administered. The patient is monitored for one (1) hour post-procedure, and if all vital signs are stable while meeting discharge criteria, the patient is released to return home.

The procedure lasts approximately one (1) hour. Then afterwards, the patient is free to return home along side his/her caregiver with recommendations for continuing care and returning to normal activities.

### Pre-Procedure:

Pre-procedural preparations include determining immediate blood alcohol level. These levels should generally be negligent before proceeding with the invention procedure. If blood alcohol level is elevated, recommendations are made to address immediate treatment and the patient is advised to return when successful abstinence has been attained. This may require professional intervention. The patient undergoes a written and visual assessment on site to determine accurate degree of addiction/abuse. History and physical status of the patient may include one or more of the following:
Medical history of the patient;
Identification and demographics including name, age, height, and weight;
Past medical history of the patient including major illnesses, surgeries/operations, current and/or chronic illnesses;
Family or childhood illnesses of the patient, previous personal and/or family history of problems associated with delivery of anesthesia;
Social history including tobacco and alcohol use;
Drug history of the patient including regular and acute medication use, including over the counter (OTC), prescribed, herbal medicines and therapies, and specifically a history of opiate use both prescribed and recreational;
Drug allergies of the patient are identified; and
Insurance information of the patient in the event that the patient may require hospital attention.

Blood is drawn from the patient for lab evaluation including blood alcohol level. Based on the patient's previous medical and physical evaluations, further cardiac evaluation may be warranted and an ECG and/or stress test may be recommended. Psychological and medical evaluation of the patient is generally conducted. Psychological evaluation of the patient is typically done by a qualified addiction treatment therapist and all medical information is reviewed. Post procedure instructions for family/friend caregiver of the patient are reviewed. Medications are drawn up and labeled for the procedure. A physical examination of the patient is performed.

Pre-Procedure Assessment of the Patient with a Physician (on the Procedure Date):

The physician will perform individual, personal interview with the patient designed to verify previously obtained information useful in formulating adequate treatment and recovery plan for the patient. Review will include, but not be limited to all of the above mentioned information in the pre-procedure. Additionally, more information may be gathered including one or more of the following:
Neurological clinical examination of the patient including consciousness, awareness, brain, vision, and others indicated by the physician; and
Description of the process to the patient—this process is to review all available data with reference to the patient's history, and description of the anticipated procedure by the physician to the patient with the patient to confirm the patient's and/or legal representative's understanding of the planned procedure.

All lab test results and any additional required tests are reviewed by the interviewing physician. Additionally bio/psycho/social assessment prior to therapy with potential follow-up relapse prevention (“continuing care”) if determined necessary by addiction therapist may be warranted. The patient will be encouraged to attend group therapy session(s) either online or in person after the procedure.

### Tests and Preparations Prior to the Procedure (on the Procedure Date):

One of more of the following tests is performed on the patient:
Alcohol urine test;
Breathalyzer;
Baseline blood levels of disulfiram via a Zenalyser®;
Urine β-hCG will be done for all women of childbearing age; and
A seven (7) panel drug test.

An intravenous line is started with a multi-vitamin preparation. If requested by the patient, an intravenous sedative is given. Vitals signs of the patient are taken and recorded. Blood pressure, ECG, SaO2, and respirations of the patient are continuously monitored during the procedure.

### Administration of Medications During the Procedure

500 mg of naltrexone palmitate is administered intramuscularly to block the effects of alcohol and control cravings. Next, 1 to 2 grams of disulfiram is administered intramuscularly to prevent the use of alcohol.

Recovery and Discharge of the Patient after the Procedure:

After the procedure, patient post-procedure evaluation begins and includes one or more of the following:
Measuring of vital signs of the patient for one (1) hour post-procedure;
Intravenous line is discontinued once the patient is ready for discharge;
Recommendations for continuing care are discussed and reviewed with the patient and caregiver, if applicable; and
Evaluation or post-procedure recommendations for necessary anxiolytics/benzodiazepine are discussed and if needed, prescriptions are written for the patient.

The patient is discharged alongside a caregiver with recommendations for continuing monthly intramuscular injections, ongoing testing via Zenalyser® as directed or recommended, multivitamin prescriptions, and recommendations for continuing care (see below). Scheduled dates for subsequent monthly intramuscular injections are confirmed with the patient prior to discharge. It has been found that long term success rates are higher with continued treatment (e.g. monthly intramuscular injections) and psychological counseling.

“Continuing Care” of the Patient after the Procedure:

After the procedure, continued care and treatment of the patient can include one or more of the following:
Bio-psycho-social assessment of the patient
Interpretive Summary;
Significant other/family education;
Medical and alternative support therapies for the patient; and,
An individualized aftercare plan for the patient.

Needs Assessment and various screening tests, such as MAST (Michigan Alcohol Screening Test) can be done at the discretion of an addiction specialist. The patient will generally have a repeat assessment in 12 months. Some of the aforementioned forms of care are descried in greater detail below.

### Bio-Psycho-Social Assessment of the Patient Prior to the Procedure:

Prior to the procedure, professionals will engage in confidential one to one interviews with the patient and caregiver/significant other. This interview is designed to gain crucial information regarding addiction history to enable the professionals and physicians to design a customized recovery aftercare plan. Such an assessment of the patient can include one or more of the following:
Patient self assessment;
Review of presenting problems;
Medical conditions of the patient;
Substance abuse history of the patient;
Employment status of the patient;
Family of origin assessment; and
Support systems for the patient.

### Interpretative Summary:

Professionals will summarize all of the assessment information, meet with the physician and together, they will plan an alcohol cessation and aftercare plan for the patient.

### Significant Other/Family Education:

Significant others and family members are greatly impacted by a loved one's addiction (i.e., the patient's addiction). A goal of the education program is generally to increase the understanding of the impact that the addictive behavior has on all significant relationships and to provide tools that will support the patient's return to an alcohol-free lifestyle. These tools can include one or more of the following:
Addiction education;
Family roles education;
Coping strategies for changing unhealthy behavior patterns of the patient; and
Developing personal recovery plan for significant other/family members of the patient.

During the invention procedure, addiction therapists can meet with the patient's loved ones to provide them with these valuable tools.

### Medical and Alternative Support Therapies for the Patient:

Medical aftercare therapies can address any underlying conditions present at the time of the invention procedure. These therapies can help diminish the symptoms without the use of traditional addictive drugs.

Individualized Aftercare Plan for the Patient after the Procedure:

The aftercare plan can include one or more of the following:
Weekly, individual mobile or internet sessions with a licensed addiction therapist enabling one to one contact with a therapist, scheduled within the patient's time constraints;
Weekly confidential and anonymous online group sessions, allowing the patient to participate in a therapist facilitated group with other recovering abusers (e.g. other patients);
Weekly questionnaires electronically transmitted from a therapist directly to the patient can keep the therapist apprised of the patient's recovery progress. If any recovery problems are indicated in the patient's responses, the therapist will recognize them and can initiate immediate relapse prevention interventions to assist the patient.

The invention procedure has shown excellent and long term success rates in a variety of patients.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, it is to be appreciated that different, special, and/or unexpected results may be obtained from each member of the respective Markush group independent from all other Markush members. Each member of a Markush group may be relied upon individually and or in combination and provides adequate support for specific embodiments within the scope of the appended claims.

It is also to be understood that any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The present invention has been described herein in an illustrative manner, and it is to be understood that the terminology which has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of independent and dependent claims, both singly and multiply dependent, is herein expressly contemplated.

What is claimed is:

1. A method of alcohol cessation and treatment for a patient, said method comprising the steps of:
administering a multi-vitamin to the patient;
administering a medication group to the patient; and
monitoring the patient.

2. The method as set forth in claim 1 wherein the step of administering the medication group is further defined as injecting the medication group into at least one muscle of the patient.

3. The method as set forth in claim 1 wherein the medication group comprises a therapeutically effective amount of an anti-addictive agent.

4. The method as set forth in claim 3 wherein the anti-addictive agent is naltrexone palmitate.

5. The method as set forth in claim 3 wherein the therapeutically effective amount of is about 500 milligrams (mg).

6. The method as set forth in claim 3 wherein the medication group further comprises a therapeutically effective amount of an inhibitor of alcohol dehydrogenase.

7. The method as set forth in claim 6 wherein the inhibitor is disulfiram.

8. The method as set forth in claim 6 wherein the therapeutically effective amount is from about 1 to about 2 grams (g).

9. The method as set forth in claim 1 wherein the multi-vitamin is administered to the patient intravenously.

10. The method as set forth in claim 1 wherein the step of monitoring the patient is further defined as monitoring vital signs of the patient and wherein the step of monitoring vital signs of the patient is contemporaneous with the step of administering the medication group to the patient.

11. The method as set forth in claim 1 further comprising the step of measuring metabolite gases of an inhibitor of alcohol dehydrogenase in the patient's breathe before and/or after the step of administering the medication group to the patient.

12. The method as set forth in claim 1 further comprising the step of administering a sedative to the patient prior to the step of administering the medication group to the patient.

13. The method as set forth in claim 1 wherein the patient is an alcohol abuser.

14. A method of alcohol cessation and treatment for a patient, said method comprising the steps of:
injecting a first injection solution into the patient; and
injecting a second injection solution separate and different from the first injection solution into the patient;
wherein the first injection solution comprises a therapeutically effective amount of an anti-addictive agent and the second injection solution comprises a therapeutically effective amount of an inhibitor of alcohol dehydrogenase.

15. The method as set forth in claim 14 wherein the anti-addictive agent is naltrexone palmitate.

16. The method as set forth in claim 15 wherein the naltrexone palmitate is present in an amount of about 200 milligrams (mg) per milliliter (ml) of the first injection solution.

17. The method as set forth in claim 16 wherein about 500 mg of naltrexone palmitate is injected into the patient via the first injection solution for administering the naltrexone palmitate to the patient.

18. The method as set forth in claim 14 wherein the inhibitor is disulfiram.

19. The method as set forth in claim 18 wherein about 1 to about 2 grams (g) of disulfiram is injected into the patient via the second injection solution for administering the disulfiram to the patient.

20. A method of alcohol cessation and treatment for a patient, said method comprising the steps of:
injecting a first injection solution into the patient; and
injecting a second injection solution separate and different from the first injection solution into the patient;
wherein the first injection solution comprises about 500 milligrams (mg) of naltrexone palmitate and the second injection solution comprises from about 1 to about 2 grams (g) of disulfiram.
